(19) United States
(12) Patent Application Publication
(10) Pub. No.: US 2006/0044509 A1
(43) Pub. Date: Mar. 2, 2006

(54) Device and method for adjusting a position of an eyeglass lens relative to the position of a pupil

(76) Inventors: Ole Fluegge, Bartholomae (DE); Matthias Kubitza, Aalen (DE)

Correspondence Address: HARNESS, DICKEY & PIERCE, P.L.C., P.O. BOX 828, BLOOMFIELD HILLS, MI, 48303, US

(21) Appl. No.: 11/188,234

(22) Filed: Jul. 22, 2005

Related U.S. Application Data
(63) Continuation of application No. PCT/EP04/00600, filed on Jan. 24, 2004.

(30) Foreign Application Priority Data
Jan. 28, 2003 (DE) 103 04 185.0

(51) Int. Cl. A61B 3/14; A61B 3/10; A61B 3/00

(52) U.S. Cl. 351/208; 351/205; 351/246

(57) ABSTRACT

An apparatus and a method are used for adapting a position of at least one spectacle lens of a spectacle relative to the position of a pupil of an eye of a person, the eye being associated to the spectacle lens. The apparatus comprises an illuminating device for an eye area of the person wearing a spectacle frame that is not yet fitted with lenses. Further, at least one camera for generating an image of the eye area is provided. The position of the pupil within the image is marked. The illuminating device has at least one light source operating within a wavelength range the light of which being reflected by the retina of the eye at a high ratio of reflection. The camera in its sensitivity is optimized to the wavelength of the light emitted by the light source.

### CROSS REFERENCE TO OTHER APPLICATIONS

The present application is a continuation of pending International patent application PCT/EP2004/000600, filed Jan. 24, 2004 which designates the United States and was published in German, and which claims priority of German patent application 103 04 185.0, filed Jan. 28, 2003. The disclosure of the above application is incorporated herein by reference.

### FIELD OF THE INVENTION

The invention is generally related to the field of adapting spectacle lenses to persons.

More specifically, the invention is related to an apparatus for adapting a position of at least one spectacle lens of a spectacle relative to the position of a pupil of an eye of a person, the eye being associated to the spectacle lens, comprising an illuminating device for an eye area of the person wearing a spectacle frame that is not yet fitted with lenses, at least one camera for generating an image of the eye area, and means for marking a position of the pupil within the image.

Further, the invention is related to a method of adapting a position of at least one spectacle lens of a spectacle relative to the position of a pupil of an eye of a person, the eye being associated to the spectacle lens, in which an eye area of the person wearing a spectacle frame that is not yet fitted with lenses is illuminated by means of a first light source, and an image of the eye area is generated, and the position of the pupil is marked within the image.

An apparatus and a method of the type specified before are known, for example from a device “Video Infral System II” of the applicant.

### BACKGROUND OF THE INVENTION

For adapting a spectacle, in particular a spectacle with progressive power lenses, an optometrist must determine the position of the pupil centers of the customer relative to the spectacle frame that shall be used, when the customer assumes a normal posture of his/her head and body. When doing so, one must take care that the position of the pupil centers is determined at a moment in time when the customer looks straight ahead and does not look to the side, for example because the optometrist manipulates in the vicinity of his/her eyes.

In prior art systems of the type specified at the outset this measurement is made from a longer distance of for example five meters. The spectacle frame that is not yet fitted with lenses is put on the customer, and the eye area of the customer is measured from the before-mentioned long distance. For that purpose, the prior art systems utilize a video camera. The image of the customer's eye area is recorded and displayed on a computer screen. By means of conventional cursor operations the optometrist can mark the pupil centers within the image, as well as certain reference lines for the position of the spectacle frame.

A disadvantage of the prior art systems is that faulty measurements relating to the exact position of the pupil centers may occur when the recorded image has only a low contrast in the pupil area, with the result that the optometrist is unable to exactly locate and mark the pupil centers within the video image. This holds true in particular for customers with dark irises from which the pupils contrast only little. If the general illumination within the examination room is set very bright, then a natural narrowing of the pupils by the customer will additionally occur.

In an apparatus of the type specified at the outset, as is known from DE 100 33 983 A1, an annular light source of undisclosed design is used for illuminating the person and the spectacle frame, resp. In another such apparatus according to DE 88 12 095 U1 a light source of likewise undisclosed design is used, the light source being shown as an incandescent bulb. Finally, FR 2 663 528 A3 discloses another such apparatus utilizing a spot light of likewise undisclosed design.

These prior art apparatuses, therefore, also use light sources emitting normal light, i.e. white ambient light.

### SUMMARY OF THE INVENTION

It is, therefore, an object underlying the invention, to further improve an apparatus and a method of the type specified at the outset such that the exact position of the pupils of a person relative to a spectacle frame may also be determined in cases where only low contrast images of the person's pupils may be generated under conventional general illumination conditions.

The measurement shall be adapted to be exact to an extent that the position of the pupil centers is determined and marked by the system itself, so as to avoid a manual marking of the positions by the optometrist, together with all sources of errors associated therewith.

In an apparatus of the type specified at the outset this object is achieved in that the illuminating device has at least one light source operating within a wavelength range the light of which being reflected by the retina of the eye at a high degree of reflection, and that the camera in its sensitivity is optimized to the wavelength of the light emitted by the light source.

In a method of the type specified at the outset this object is achieved in that the eye area is illuminated with light of a wavelength range being reflected by the retina of the eye with a high degree of reflection, and that the image of the eye area is generated with a sensitivity optimized to the wavelength of the light.

The object underlying the invention is, thus, entirely solved.

By illuminating the eye portion of the person with a light from the specified wavelength range the retina of the eye is caused to reflect such light so that it distinguishes from the surrounding iris at high contrast. The use of a camera optimized for this wavelength has the advantage that particular clear and high contrast images may be obtained even at low light intensity.

The optometrist is therewith enabled to reliably determine the position of the pupil centers by manual marking with the cursor. However, it is still more advantageous when this manual operation is replaced by an automatic determination of the pupil centers by means of conventional image processing techniques. By doing so, all additional sources of errors are eliminated which are associated to any manual operation. The method, in contrast to conventional methods, further, becomes so simple in its execution that it may be repeated several times for entirely eliminating falsifying influences. Such influences may be, for example, an accidental convergent eye movement of the person.

Although from DE 196 49 542 C2 there is already known a method for measuring a pupil, in which the pupil is illuminated with infrared light, the use of infrared (non-visible) light in this context has the sole purpose to exclude molestation of the patient under examination. This application does not take into account a reflection from the retina that would make the entire pupil shine. Due to the angle between the direction of illumination and the direction of observation, such reflection would not even occur.

U.S. Pat. No. 5,150,137 discloses a system for functional measurements on pupils. In one disclosed embodiment (FIG. 34) an apparatus is provided, in which an infrared light diode emits a measuring light along the same optical axis, along which the observation device is directed onto the eye. In this prior art system, however, only one eye is examined at a small distance so that the problems discussed at the outset do not occur.

In preferred embodiments of the apparatus according to the invention the light source emits light in the red over to the infrared range, wherein the light source, preferably, is a light diode or an array of light diodes.

These measures have the advantage that apparatuses with high reliability and low production costs may be manufactured with commercially available components.

The illumination device advantageously comprises a lens for focussing the light emitted by the light source, as desired.

In an embodiment of the invention the camera comprises a plurality of color channels, and image signals of the one color channel coming spectrally closest to the light emitted by the light source, in particular of the red channel, are adapted to be separately processed to images.

This measure has the advantage that commercially available video cameras may be used that comprise a red channel, such that the respective image signals may separately be processed to images in which the red light remitted by the retina appears particularly well.

As an alternative, one may also provide at least two cameras, one of which being optimized in its sensitivity to the wavelength of the light emitted by the light source.

This measure, too, has the advantage that images with normal light as well as images with the mentioned particular light may be generated, as will be described in further detail below.

In particularly preferred embodiments of the invention the camera and the light source are arranged towards the eye essentially along the same optical axis. When doing so, the camera and the light source are inclined with respect to each other by less than 2°, preferably less than 1°.

This measure has the advantage that the light remitted by the retina may particularly well be received in the camera because at least with persons having defective vision the light irradiated into the eye is reflected or remitted, resp., by the retina as a narrow pencil of rays with small divergence.

In a practical realization of this embodiment a beam splitter is arranged within the beam path between the camera and the eye for coupling in the light of the light source, the light of the beam splitter being reflected in the direction of the optical axis of the camera away from the latter.

This measure has the advantage that the above-mentioned coaxial orientation of the camera on the one hand and of the light source light on the other hand is achieved with simple design means.

In a preferred refinement of this embodiment the beam splitter has a degree of reflection of below 50%, preferably of between 8% and 40% for the light remitted by the eye.

Although this measure is energetically not optimal since a half mirror with 50% is conventionally considered optimal, the above-mentioned range of the degree of reflection is nevertheless preferred for practical reasons, wherein in a still further refinement of this embodiment the degree of reflection for wavelengths outside the wavelength range of the light emitted by the illuminating device is still lower.

These measures have the advantage that also the spectacle frame in front of the person's face may be detected reliably and that, moreover, false measurements are avoided as may occur with persons having a dark colored skin.

Further, within the context of this embodiment, it is preferred when a light trap is arranged on the side of the beam splitter opposite the light source.

This measure has the advantage that the light emitted by the light source is reliably absorbed to the extent as it is not deflected away from the camera at the beam splitter.

In another particularly preferred group of embodiments additional light sources are provided outside the optical axis, the additional light sources being directed to the eye area.

This measure has the advantage that the invention may also be used for persons with a strong defective vision with which the light of the light source irradiated along the optical axis is not reflected as a narrow, i.e. low divergent pencil of rays. By means of the discussed measures a signal remitted by the retina is received by the camera, the orientation of which being unchanged along the above-mentioned axis, also in the case of such a strong defective vision (in particular myopia).

In that case it is particularly preferred when the additional light sources are arranged equally, i.e. ring-shaped, about the optical axis and are inclined with respect to the latter.

For the apparatus according to the invention it is preferred, as in the prior art, when the beam path between the camera and the illuminating device on the one hand, and the eye on the other hand, has a length of several meters, preferably of between two and eight meters.

In case the local circumstances within the examination room do not allow such distances, it is, in a conventional manner, preferred when the beam path is folded.

In another group of embodiments there is provided a general illumination for the eye area besides the illuminating device, and means are provided for controlling the camera, such that the camera alternately records a first image with the general illumination only and the light source being switched off, and a second image with the light source being switched on.

This measure has the advantage that in separate operations a first, normal image of the eye area of the person on the one hand and, on the other hand, a second image may be recorded on which the pupils shine in contrast to the first image. The position of the pupils may be easily found in a differential image of the two images with the help of image processing methods, and may then be determined exactly in the second image with the shining pupils.

In a preferred variation of this embodiment the camera records the second image with the general illumination being switched off.

This measure has the advantage that an interference during the recording of the second image through the general illumination is avoided.

In this context it is particularly preferred when the camera records the first and the second image immediately one after the other, in particular when the camera is a so-called “interlaced” camera, and that the camera records the first and the second image as half-images of a full-image.

These measures have the advantage that one may fall back to courses of action, known per se, namely the so-called “interlaced” methods.

The same advantages as explained before with regard to embodiments of the apparatus according to the invention also hold true mutatis mutandis for embodiments of the method according to the invention.

Further advantages will become apparent from the description and the enclosed drawings.

It goes without saying that the features mentioned above and those that will be explained hereinafter may not only be used in the particularly given combination but also in other combinations or alone without leaving the scope of the present invention.

### BRIEF DESCRIPTION OF THE DRAWINGS

Embodiments of the invention are shown in the drawings and will be explained in further detail in the subsequent description. In the Figures:

FIG. 1 shows a first embodiment of an apparatus according to the invention, in a side elevational view and highly schematic;

FIG. 2 shows a variation of the embodiment of FIG. 1;

FIG. 3 is an image of an eye area of a person, as may be recorded with the apparatuses of FIG. 1 or 2;

FIG. 4 shows a detail for explaining the function of the apparatus of FIG. 2;

FIG. 5 shows another detail for explaining the operation of the apparatus of FIG. 2 in connection with FIG. 4;

FIG. 6 is a block diagram showing an electronic control for the apparatuses of FIG. 1 or 2; and

FIG. 7 is a pulse diagram for explaining the block diagram of FIG. 6.

### DETAILED DESCRIPTION OF THE PREFERRED EMBODIMENTS

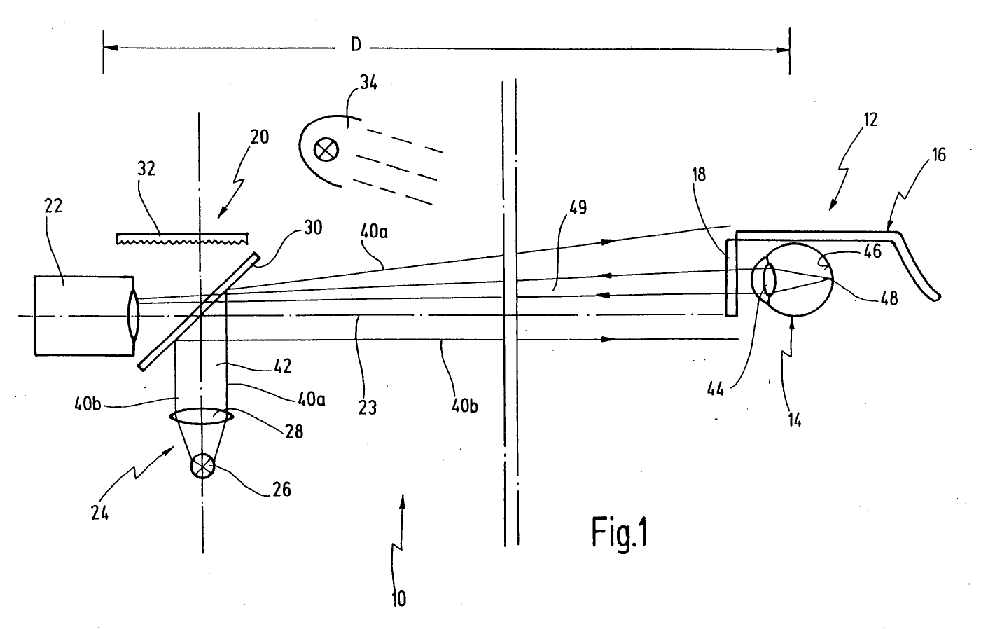

In FIG. 1 reference numeral 10 as a whole indicates an apparatus for adapting a position of at least one spectacle lens, in particular of a progressive power lens, of a spectacle relative to the position of a pupil of an eye of a person, the eye being associated to the spectacle lens.

In FIG. 1 the person as a whole is indicated at 12, only an eye 14 and a spectacle 16 or spectacle frame 18, resp., being shown.

A recording system indicated as a whole at 20 is located at a distance D of several meters, preferably two to eight meters.

Recording system 20 comprises a camera 22, the optical axis of which is designated with reference numeral 23.

An illumination device 24 is provided under a right angle relative to axis 23. Illumination device 24 comprises a light source 26, in particular a light diode (LED) operating in the red or the infrared range. Light source 26 has a lens 28 associated thereto. Light source 26 is directed onto a beam splitter 30. A light trap 32 is arranged on the opposite side of beam splitter 30.

Finally, recording system 20 comprises a general illumination device 34 with conventional white light.

In FIG. 1, reference numerals 40a and 40b indicate marginal rays of light 42 emitted by light source 26. Light 42 and marginal rays 40a, 40b, resp., are reflected at beam splitter 30 and are directed onto eye 14 of person 12. Light 42 enters eye 14 via an eye lens 44 and impinges on a retina 46 on which an image 48 is generated. If person 12 has normal vision, image 48 is a focussed image, whereas if person 12 has defective vision, an unfocussed image is generated, as will be explained.

Reference numeral 49 designates a light being remitted by retina 46. Light 49, in turn, impinges on beam splitter 30 and partially falls into camera 22.

Beams splitter 30 is preferably configured as a partially transparent mirror. It consists of a transparent plane-parallel plate, e.g. made from glass, one side of which being unprocessed or partially reflective and the other side of which being dereflected.

The mirror may have a degree of reflection of 50%. With that selection of the degree of reflection a maximum of remitted light 49 would be directed into camera 22. Light 42 emitted by light source 26 namely is reflected with the same degree of reflection by the mirror, is directed towards eye 14 and is there remitted. Remitted light 49 with its fraction being transmitted through beam splitter 30 falls into camera 22.

From an energetic point of view a degree of selection of 50% would, therefore, be optimal. For practical reasons, however, one strongly deviates from that value and uses a degree of reflection being of the order of between 8% and 40%.

Moreover, it is preferred to select a mirror coating having advantageously a still lower degree of reflection for wavelengths at which light 42 has no or a small intensity. Degrees of reflection being smaller than those 50% mentioned are, moreover, particularly helpful because one must also detect the spectacle frame 18 in front of the face of person 12. If person 12 has a dark colored skin, the advantage of this measure is particularly great.

As has already been mentioned, light source 26 preferably is a light diode operating in the red or the infrared range. Instead of one single light diode one may alternately also use a bundle of such diodes, however, lens 28 would then have to be configured as a corresponding honeycomb structure, as known per se.

Light trap 32 being only schematically indicated in FIG. 1 is provided for absorbing light 42 having run through beam splitter 30 unreflected. One might use a black cardboard, a soot-covered sheet metal or a surface to which a black velvet is glued as light trap 32. Such a light trap might also be configured as a so-called “black bag”.

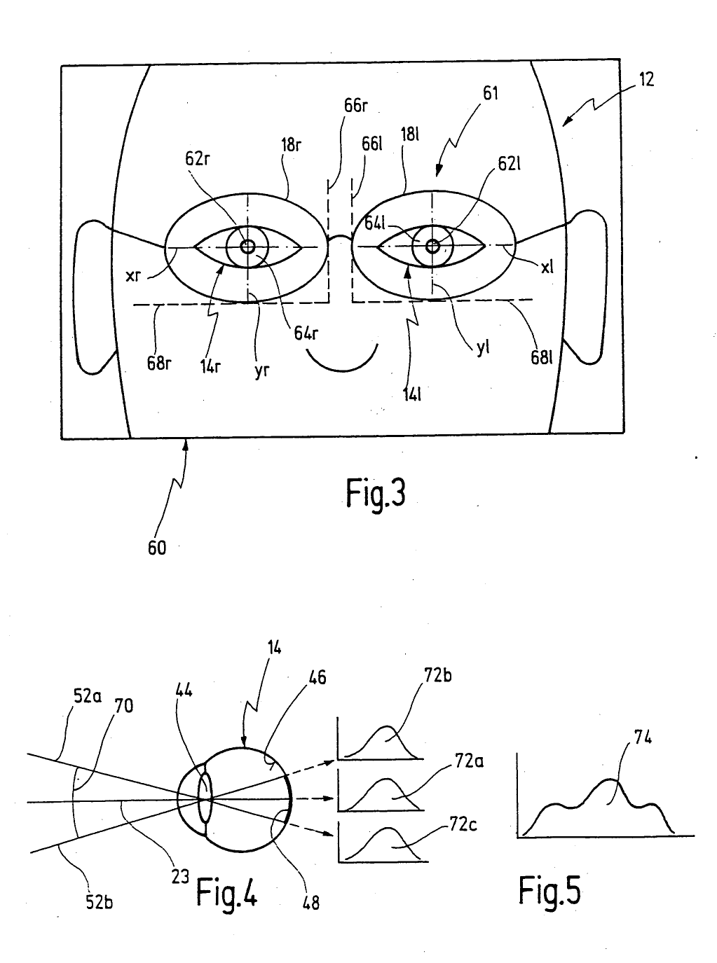

FIG. 3 shows an image 60 recorded by camera 22. One can see an eye area 61 of person 12. A right pupil and a left pupil of person 12 are designated 62r, 62l, a respective corresponding iris 64r, 64l. The center of each iris 64r, 64l is inserted in FIG. 3 as a cross of two dash-dot lines.

Reference lines for spectacle frame 18r and 18l, resp., are entered as vertical lines 66r, 66l and as horizontal lines 68r, 68l.

One may clearly see from FIG. 3 that the exact position of the center of each iris 64r, 64l as well as the exact position of spectacle frames 18r and 18l may be also automatically detected from image 60 by means of conventional image processing methods. In any event it is manually possible to simply identify these points and lines, resp., by means of a cursor and to mark same in image 60.

Due to the selected wavelength of light 42 retina 46 behind pupils 62r and 62l shines brightly such that pupils 62r, 62l clearly contrast from the respective surrounding iris 64r and 64l, resp. This holds also and particularly true when iris 64r, 64l is relatively dark by itself.

FIG. 4 shows the circumstances with a person 12 having accommodated on a short distance, e.g. until point 70, in particular because person 12 is short-sighted. At point 70 there is a real image of retina 46 within eye 14.

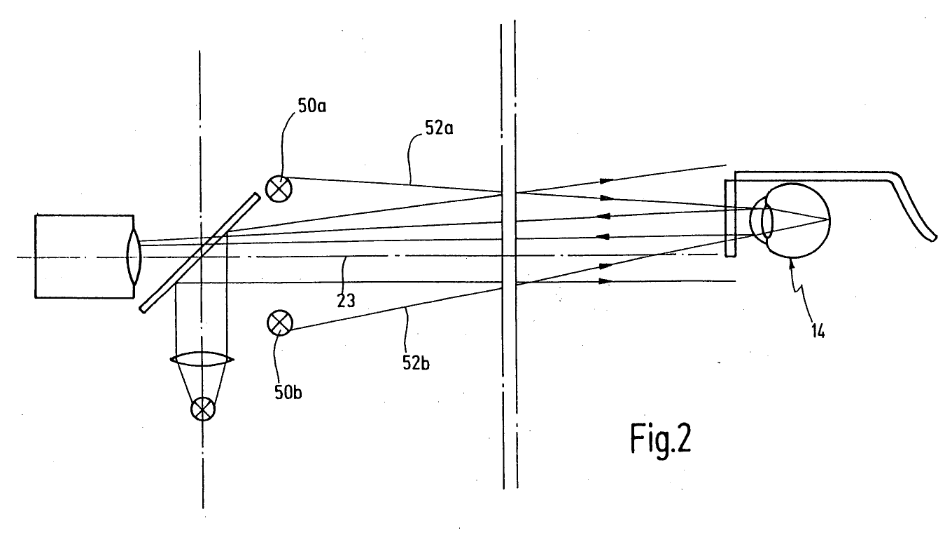

In order to be able to conduct a successful measurement also in that case, the embodiment of FIG. 2 is used in which additional light sources 50a, 50b are arranged about axis 23, in particular in a ring-shaped configuration. Marginal rays 52a, 52b shown in FIG. 2 characterize the light emitted by additional light sources 50a, 50b. This light runs towards the pupil centers of the person. Due to the defective vision unfocussed images of additional light sources 50a, 50b are generated on retina 46 of eye 14. The intensity distributions around the geometric projection points along marginal rays 52a, 52b are schematically depicted in partial illustrations 72a through 72c at the right hand side of FIG. 4. Of course, in FIG. 4 the angles between marginal rays 52a, 52b and axis 23 are shown highly exaggerated and much bigger than in reality.

As one can take from partial illustrations 72a through 72c, the margins of external intensity distributions 72b, 72c overlap with central intensity distribution 72a such that, seen as a whole, an overlayed intensity distribution results as again shown separately in FIG. 5 at 74.

Taken altogether, an extended and unfocussed image shines on retina 46 being significantly brighter than the unfocussed partial image of the central intensity distribution 72a alone. Eye lens 44 creates the real air image of retina 46 on which unfocussed image 48 shines.

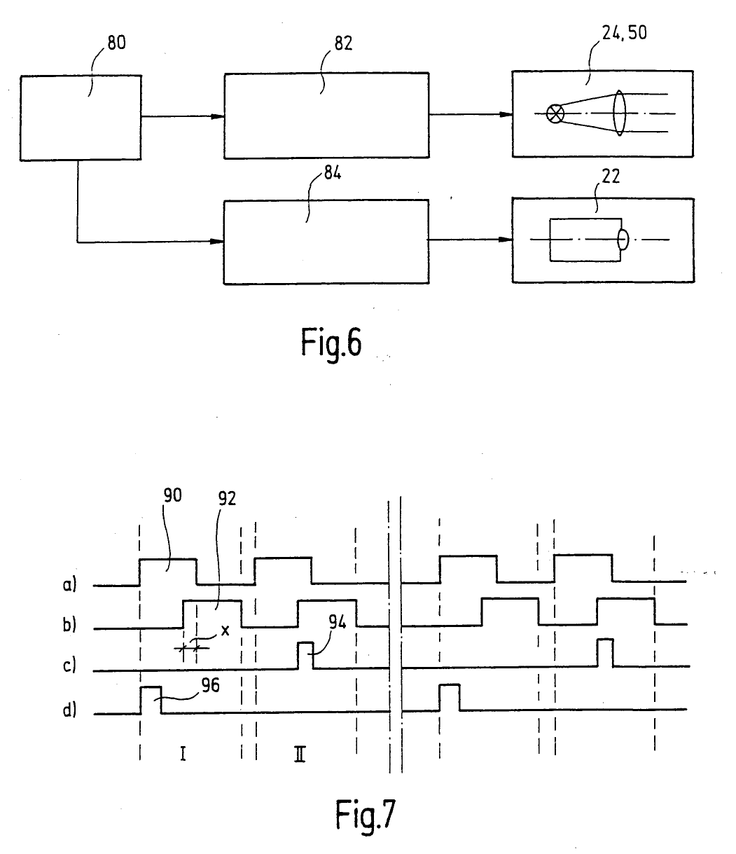

FIG. 6 shows a schematic block diagram for controlling the apparatus according to the invention, in a preferred embodiment.

A computer 80 is connected to a control 82 device for light sources 26 and 50. Computer 80, further, is connected to an image acquisition unit 84 to which camera 22 is coupled.

During a measurement events occur in a time sequence as depicted in FIG. 7, for example. FIG. 7 shows the circumstances with a conventional camera in the so-called “interlaced” method. In that method two half-images are generated one after the other which may be combined to be a full-image. However, it goes without saying that the present invention may likewise be used with cameras that may only be operated in the full-image mode.

In FIG. 7, in lines a) and b) those time intervals in which the camera is sensitive for the half-images (integration time interval) are depicted as pulses 90 and 92 for the two half-images. Lines c) and d), in contrast, show illuminating pulses 94 and control pulses 96.

The measurement is initiated with a control pulse 96, whereupon a first half-image 90 and a second half image 92 are generated. One can clearly take from FIG. 7 that the two half-images have a certain range x of overlap, i.e. a time interval during which both half-images are sensible to light.

In a first cycle I the two half-images are recorded with the general illumination device 34 being switched on.

In the subsequent cycle II light sources 26 and 50 are switched on for a short period of time as indicated with light pulse 94, for example just at the moment in time when both half-images are sensible to light.

Computer 80 now has two half-images from cycle I with only the general illumination device switched on, and has two half-images from cycle II with light sources 26 and 50 switched on. During cycle II general illuminating device 34 may be switched off.

By doing so one obtains two full-images, the first one of which having been recorded only with the general illuminating device 34, and the second with light sources 26 and 50.

As an alternative also such a sequence may be generated within two half-images, wherein the first half-image is recorded only with the general illuminating device and the second half-image only with or in addition with the light source. Insofar, the invention is no subject for limitations.

From the images so recorded (cf. FIG. 3) the desired positions may now be determined in the already mentioned manual or automatic manner.

1. An apparatus for adapting a position of at least one spectacle lens of a spectacle relative to a position of a pupil of an eye of a person, said eye being associated to said spectacle lens, comprising an illuminating device for an eye area of said person wearing a spectacle frame that is not yet fitted with lenses, at least one camera for generating an image of said eye area, and means for marking a position of said pupil within said image, wherein said illuminating device has at least one light source operating within a wavelength range, a light of which being reflected by said retina of said eye at a high degree of reflection, and said camera in its sensitivity being optimized to said wavelength of said light emitted by said light source.

2. The apparatus of claim 1, wherein said light source emits light in a red over to an infrared range.

3. The apparatus of claim 1, wherein said light source is a light diode.

4. The apparatus of claim 1, wherein said light source is an array of light diodes.

5. The apparatus of claim 1, wherein said illuminating device comprises a lens.

6. The apparatus of claim 1, wherein said camera comprises a plurality of color channels, image signals of said one color channel coming spectrally closest to said light emitted by said light source, in particular of said red channel, being adapted to be separately processed to images.

7. The apparatus of claim 1, wherein at least two cameras are provided, one of which being optimized in its sensitivity to said wavelength of said light emitted by said light source.

8. The apparatus of claim 1, wherein said camera and said light source are arranged towards said eye essentially along a same optical axis.

9. The apparatus of claim 8, wherein said camera and said light source are inclined with respect to each other by less than 2°.

10. The apparatus of claim 8, wherein said camera and said light source are inclined with respect to each other by less than 1°.

11. The apparatus of claim 8, wherein within said beam path between said camera and said eye a beam splitter is arranged for coupling in said light of said light source, said light of said beam splitter being reflected in said direction of said optical axis of said camera away from the latter.

12. The apparatus of claim 11, wherein said beam splitter has a degree of reflection of below 50% for said light remitted by said eye.

13. The apparatus of claim 11, wherein said beam splitter has a degree of reflection of between 8% and 40% for said light remitted by said eye.

14. The apparatus of claim 13, wherein said degree of reflection for wavelengths outside said wavelength range of said light emitted by said illuminating device is still lower.

15. The apparatus of claim 12, wherein a light trap is arranged on a side of said beam splitter opposite said light source.

16. The apparatus of claim 10, wherein additional light sources are provided outside said optical axis, said additional light sources being directed to said eye area.

17. The apparatus of claim 16, wherein said additional light sources are arranged equally about said optical axis and are inclined with respect to the latter.

18. The apparatus of claim 1, wherein said beam path between said camera and said illuminating device on the one hand, and said eye on the other hand, has a length of several meters.

19. The apparatus of claim 1, wherein said beam path between said camera and said illuminating device on the one hand, and said eye on the other hand, has a length of between two and eight meters.

20. The apparatus of claim 19, wherein said beam path is folded.

21. The apparatus of claim 1, wherein besides said illuminating device there is provided a general illumination for said eye area, and means being provided for controlling said camera, such that said camera alternately records a first image with said general illumination only and said light source being switched off, and a second image with said light source being switched on.

22. The apparatus of claim 21, wherein said camera records said second image with said general illumination being switched off.

23. The apparatus of claim 21, wherein said camera records said first and said second image immediately one after the other.

24. The apparatus of claim 23, wherein said camera is a line camera, said camera recording said first and said second image as half-images of a full-image.

25. A method of adapting a position of at least one spectacle lens of a spectacle relative to a position of a pupil of an eye of a person, said eye being associated to said spectacle lens, in which an eye area of said person wearing a spectacle frame that is not yet fitted with lenses is illuminated by means of a first light source, and an image of said eye area is generated, and said position of said pupil is marked within said image, wherein said eye area is illuminated with light of a wavelength range being reflected by said retina of said eye with a high degree of reflection, said image of said eye area being generated with a sensitivity optimized to said wavelength of said light.

26. The method of claim 25, wherein light is emitted within a red over to an infrared range.

27. The method of claims 25, wherein said image is recorded essentially along a same axis along which said eye area is illuminated.

28. The method of claim 27, wherein said eye area is additionally illuminated from at least one direction outside said optical axis.

29. The method of claim 28, wherein said eye area is additionally illuminated from several directions by means of second light sources, wherein said directions are arranged equally about said optical axis and are inclined with respect to the latter.

30. The method of claim 25, wherein besides from said light sources said eye area is further illuminated by a general illumination, wherein, alternately, a first image is recorded with said general illumination only and said light source being switched off, and a second image is recorded with said light source being switched on.

31. The method of claim 30, wherein said second image is recorded with said general illumination being switched off.

32. The method of claim 30, wherein said first and said second image are recorded immediately one after the other.

33. The method of claim 32, wherein said first and said second image are recorded as half-images of a line full-image.
